(19) United States
(12) Patent Application Publication
(10) Pub. No.: US 2021/0338535 A1
(43) Pub. Date: Nov. 4, 2021

(54) Dual action oral waste fluid removal and hydration device

(71) Applicants: Maria Dominic, Odessa, TX (US); Olufemi Dominic, Odessa, TX (US)

(72) Inventors: Maria Dominic, Odessa, TX (US); Olufemi Dominic, Odessa, TX (US)

(21) Appl. No.: 16/865,943

(22) Filed: May 4, 2020

(51) Int. Cl. A61J 19/02; B65D 1/02; B65D 23/00; A61M 1/00

(52) U.S. Cl. CPC A61J 19/02; B65D 1/02; A61M 2210/0625; A61M 1/0062; B65D 23/00

(57) ABSTRACT

The device comprises a portable, dual-cup, twin-bottle apparatus. One of the bottles carries a fresh fluid. The other has the ability to accept mouth fluids and can include a suction actuator.

The present invention relates to a portable, dual-cup twin bottle device that is capable of providing fluids and alternatively sucking fluids from the mouth of a user. The invention allows users to avoid the unseemly practice of spittingg, while still allowing users to remove undesirable fluids from the users mouth.

### Background Art

Spitting is the act of forcibly removing fluids from the mouth of a user. The act is often done to get rid of unwanted or foul-tasting substances in the mouth or to get rid of a large buildup of saliva. Unwanted fluids or saliva in a users mouth can occur through the introduction of a foreign substance that elicits such a response, or extra-secretion by the salivary glands in a person's mouth. While practiced in many cultures and societies, in the present day with bacteria. and viruses being capable of being spread through spitting in public, many societies have banned spitting, to the point of making such an act illegal. The natural human response of extra-secretion by the salivary glands juxtaposed against societies desire to improve public health by stopping spitting causes problems for people who possess a condition resulting in saliva buildup. Examples of such people are pregnant women, tobacco users, individuals having illness, and young people such as toddlers.

There exists in the industry a need to allow people to discharge fluids from mouths while avoiding spitting, and subsequently introducing new, fresh fluid.

### BRIEF DESCRIPTION OF THE INVENTION

The present nvention has as its goal a device for accepting fluids from a users mouth, and introducing fresh fluids. The device accomplishes this goal by providing a portable, dual-cup twin bottle apparatus, whereby one bottle of the twin possesses a fresh fluid, while the second bottle of the twin has the ability to accept mouth fluids. The twin bottle design means the two bottles are attached to each other, thus a user does not have to worry about ensuring she has both bottles when traveling.

### BRIEF DESCRIPTION OF THE DRAWINGS

FIG. 1 shows twin-bottled apparatus of the present invention;

FIG. 2 shows the outward appearance of the twin-bottled apparatus of the present invention;

FIG. 3 shows the underside of the present apparatus;

FIG. 4 is an embodiment of the topside of the present apparatus.

It will be readily understood that the components of the embodiments as generally described and illustrated in the FIGs herein, may be arranged and designed in a wide variety of different configurations in addition to the described example embodiments. Thus, the following more detailed description of the example embodiments, as represented in the FIGs, is not intended to limit the scope of the embodiments, as claimed, but is merely representative of example embodiments.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

### DESCRIPTION

With reference to the Figures,

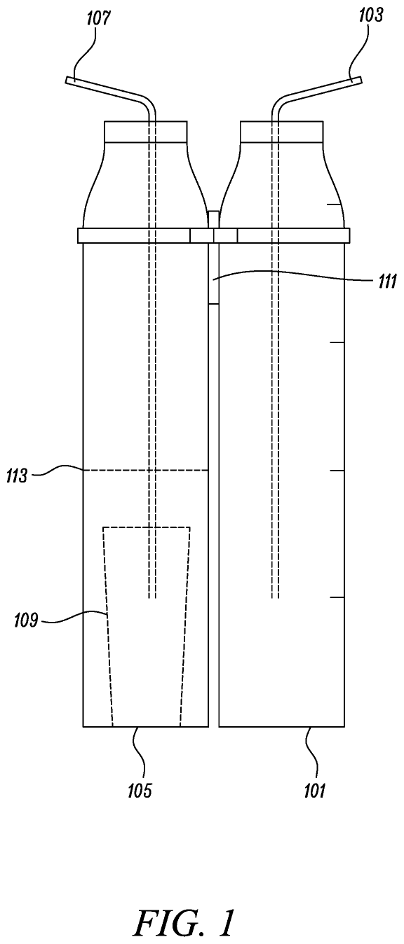

FIG. 1 is an embodiment of the present invention, including a twin-bottle apparatus 100, wherein one side of the bottle 101 is a fluid-possessing, extrusion side, and the other side of the bottle is a fluid-sucking side 105. In one embodiment, the two sides are removably connected to each other through a common anchor 111. In other embodiments, the sides may be permanently connected to each other. In still other embodiments, the sides may be connected by hook and loop attachment means, elastic bands, or snaps.

The side of the bottle that is the fluid-possessing, extrusion side 101 is an empty volume space wherein a fluid may be poured or entered therein. Any fluid is suitable for use in the present invention, however preferably the fluid is one that is vicious enough to be sucked through a straw. The amount of fluid entered can be at the discretion of the user, however, the amount of fluid must at least be above the lowest level of the straw 103. The fluid may be poured into empty volume through the removable top of that side 101 of the bottle. The straw 103 as used in the apparatus is preferably a re-usable version, and can be made of a re-usable material such as metal, paper, or plastic. The straw 103 is preferably a “bendy” or “bendable” straw, whereby the head of the straw 103 can be bent toward a desired direction of the user.

The interior of that side of the bottle 101 is preferably a metallic, non-rusting material, allowing that side of the bottle 101 to be re-usable. The side of the bottle 101 can further be an insulated flask, for example a vacuum flask. As is known in the art, the vacuum flask consists of two vessels, one placed within the other and joined at the neck. The gap between the two vessels is partially evacuated of air, creating a partial-vacuum which reduces heat conduction or convection. Heat transfer by thermal radiation may be minimized by silvering flask surfaces facing the gap but can become problematic if the flask's contents or surroundings are very hot; hence vacuum flasks usually hold contents below the boiling point of water. Most heat transfer occurs through the neck and opening of the flask, where there is no vacuum. Vacuum flasks are usually made of metal, borosilicate glass, foam or plastic and have their opening stoppered with cork or polyethylene plastic.

The other side of the bottle 105 is a fluid-sucking side, whereby a user may insert the head of the straw 107 into her mouth, and activate a suction actuator. Mouth fluids can then be sucked from the users mouth, down through the straw 107 body and into a collection cap 109. The internal of the fluid-sucking side 105 is an empty volume, possessing primarily the body of the straw 107. The straw 107 is preferably a re-usable version, and can be made of a re-usable material such as metal, paper, or plastic. The straw 107 is preferably a “bendy” or “bendable” straw, whereby the head of the straw 107 can be bent toward a desired direction of the user.

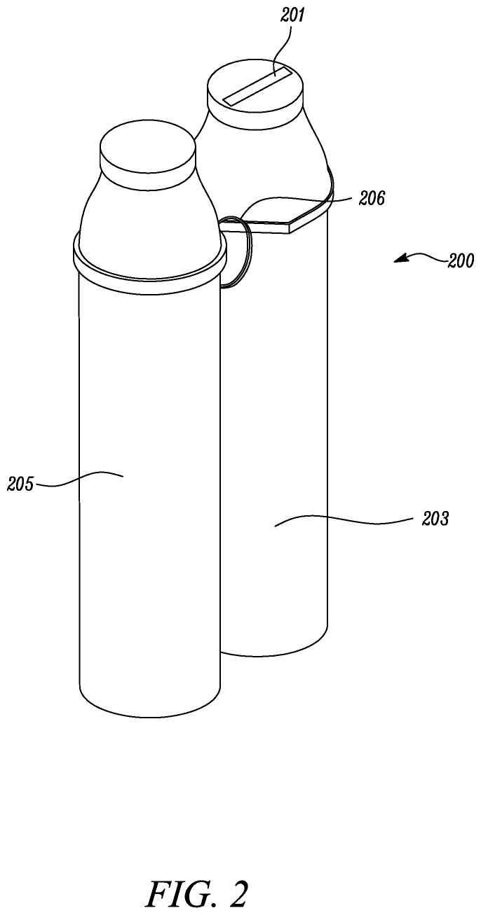

FIG. 2 is an external view of the apparatus 200 of the present invention. As shown, the apparatus 200 consists of a dual-bottle design 203/205, whereby each bottle has a cap 201. The bottles 203 and 205 are connected to each other via an attachment means 206. The attachment means 206 can be removable or permanent. If the attachment means 206 is removable, the bottles 203 and 205 may be separated, thus allowing for easy cleaning. The apparatus 200 can be a variety of sizes, ranging from 50 ml to 250 ml. In a preferred embodiment, the apparatus 200 is sized to fit with a purse, for example being approximately 100 ml in size.

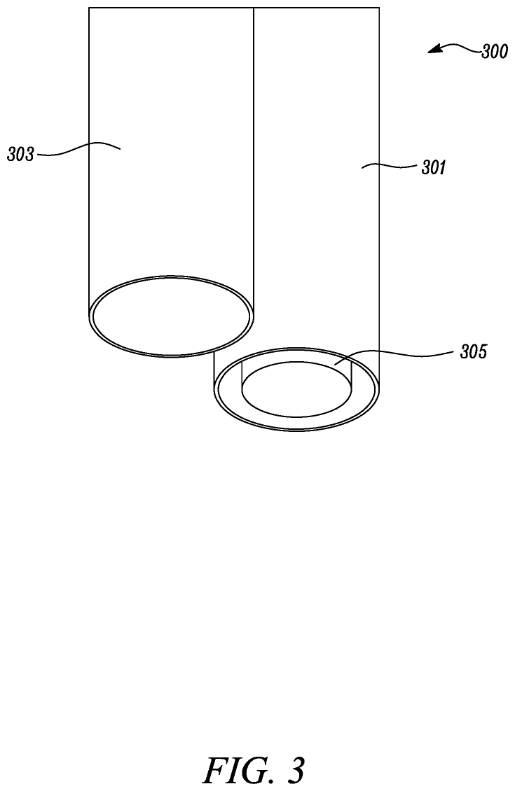

FIG. 3 is an embodiment of the present invention, whereby the bottom side of the apparatus 300 is shown. The apparatus 300, as discussed above, possesses two sides 303 and 301, whereby one side 301 is a fluid suction side. The fluid suction side 301 has on its bottom a suction actuator 305. The actuator 305 is capable of an upward and downward stroke. Upon being depressed, the actuator 305 begins its upward stroke. The upward stroke pushes air up into the straw (not shown). When the height of the upward stroke is met, the actuator 305 begins its downward stroke. The downward stroke thus sucks air into the straw. When the straw is in the mouth of a user, during the downward stroke, air and fluids can be sucked from the mouth of the user. Fluids that are sucked from the users mouth are then stored in the fluid suction side 301.

In one embodiment, the actuator 305 is removable, thus allowing sucked fluids to be removed from the fluid suction side 301. In another embodiment, the cap (not shown) of the fluid suction side 301 is removable, allowing a user to be able to flush the bottle 301.

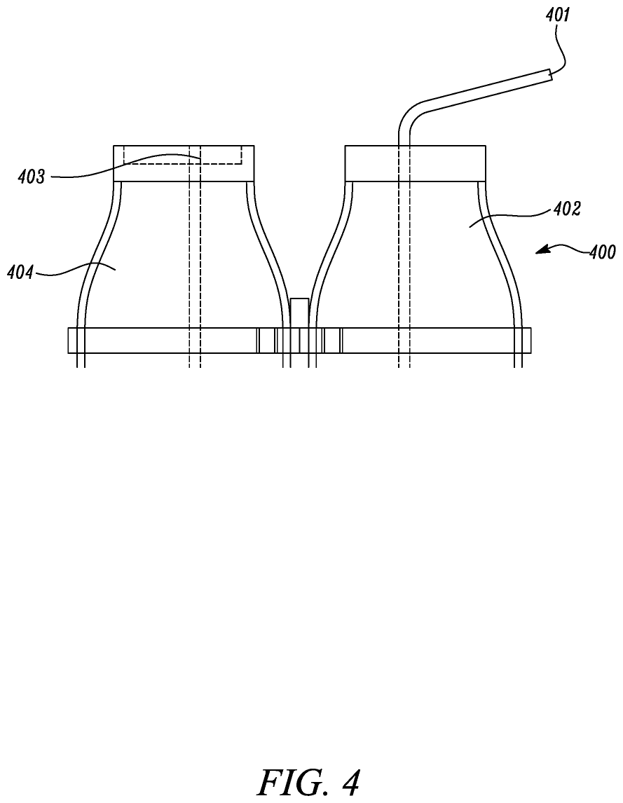

FIG. 4 is an embodiment of the top section of the apparatus 400, whereby the apparatus 400 includes a cap 404 for the second bottle and a cap 402 for the first bottle. In a preferred embodiment, the caps 404 and 402 are removable, allowing the internal volumes of the second bottle and the first bottle to be cleaned. Both the second bottle and first bottle possess separate straws 401 and 403, whereby one straw 401 is for a users to obtain fluids and the second straw 403 will allow fluids in a users mouth to be removed.

What is claimed is:

1. A device comprising:
a first bottle and a second bottle; wherein
the first bottle is configured to contain a fresh fluid and to supply the fresh fluid to the user; and
the second bottle is configured to receive a mouth fluid from the user.

2. The device of claim 1, wherein the first bottle and the second bottle are attached to each other.

3. The device of claim 1, wherein the second bottle comprises a suction actuator.

4. A method comprising:
providing a first bottle and a second bottle, wherein the first bottle is configured to contain a fresh fluid and to supply the fresh fluid to the user, and the second bottle is configured to receive a mouth fluid from the user;
supplying the fresh fluid to the user; and
receiving the mouth fluid from the user.

5. The method of claim 4, wherein the first bottle and the second bottle are attached to each other.

6. The method of claim 4, wherein the second bottle comprises a suction actuator, and wherein the receiving is performed using the suction actuator.
